(19) United States
(12) Patent Application Publication
(10) Pub. No.: US 2021/0052426 A1
(43) Pub. Date: Feb. 25, 2021

(54) FACE COVERING SYSTEMS, METHODS, AND DEVICES

(71) Applicant: Kristen Gruber Reif, Broomfield, CO (US)

(72) Inventor: Kristen Gruber Reif, Broomfield, CO (US)

(21) Appl. No.: 16/956,854

(22) PCT Filed: Dec. 22, 2018
(86) PCT No.: PCT/US18/67413

§ 371 Date: Jun. 22, 2020

Related U.S. Application Data
(60) Provisional application No. 62/609,583, filed on Dec. 22, 2017.

(51) Int. Cl. A61F 9/02; A42B 3/22

(52) U.S. Cl. CPC A61F 9/029; A42B 3/225

(57) ABSTRACT

A device, including a first attachment connected at a side of the head of a user to a piece of equipment worn by the user, and a second attachment connected at another side of the head of the user to the piece of equipment, where a covering extends between the first attachment and the second attachment

### CROSS-REFERENCE TO RELATED APPLICATION

This application is a national stage application, filed under 35 U.S.C. § 371, of International Patent Application No. PCT/US2018/067413, filed on Dec. 22, 2018, which claims the benefit of U.S. Provisional Patent Application No. 62/609,583, filed Dec. 22, 2017, each of which is incorporated by reference herein in its entirety.

### FIELD

The invention relates generally to methods and apparatuses of coverings and particularly to methods and apparatuses of face coverings.

### BACKGROUND

The present disclosure relates to apparatuses, methods, and systems of coverings.

### SUMMARY

In harsh or unfavorable environments, coverings are used to protect or shield the body. For example, in cold weather, people use face (including head) coverings to protect the skin on their face and necks from exposure to the unfavorable conditions. The term “face” as used herein includes various parts of the body, such as the cheeks, chin, ears, mouth, nose, neck and chest. Such coverings can be used by skiers, snowboarders, sledders, outdoor enthusiasts, campers, utility workers, construction workers, and others. Coverings can not only protect against cold weather, they may also be used in other types of weather, such as to protect from exposure to sun, rain, wind, hot air, etc.

The present disclosure recognizes that prior art coverings, including face coverings, have many problems. For example, they may not stay in position or they may shift positions during use, they may be too loose or tight or have portions that are too loose or too tight, they may not cover all of the area of the body that is desired to be covered by the user, and they may become unpleasant when being used in certain weather conditions. For example, many types of face coverings are designed to be adjacent to (or cover) a user's mouth and/or nose and these face coverings can act as traps for humidity from exhaled air from the user. This humidity may condense on contact with cold air, thereby moistening or freezing on the covering, and this wet or frozen portion of the covering is then adjacent to or touching the user's face, causing unpleasant feelings of cold and moisture for the user or even injuring the skin.

Also, prior art face coverings are sometimes designed so that the user must breathe through a material of the covering, or through vents or holes within the coverings. Thus, these prior art coverings may trap exhaled air of the user and thereby collect the humidity from the exhaled breath of the user. The user may have to experience unpleasant feelings of contact with dampness or cold, or the user may want to adjust the face covering to try to reduce the moisture or replace a moist or soiled part of the covering with a dry or clean part of the covering.

This can be problematic because, when performing various activities, it may not be easy or possible to constantly adjust the covering or change the covering for a new one. For example, during an activity, a user may not be able to stop the activity to adjust the face covering. Also, some activities do not allow for easy use of the hands to adjust a covering (e.g., the activity may require the use of hands or that the hands have reduced dexterity, for example, by wearing gloves or mittens) and because of this, the user may be unable to properly or easily adjust their face covering.

Further, prior art face coverings such as gaiters and balaclavas can be difficult to position or keep in place when being used with other garments (e.g., goggles, sunglasses, helmets, hats, etc.) because once one item is (or multiple items are) in position and a user attempts to put another item in position, it can cause a previous item (or multiple items) already placed in position to move out of their position(s). Embodiments of the present disclosure advantageously improve such problems.

In addition, many prior art face coverings, such as balaclavas or gaiters, are designed to fit snugly against the user's body, and in the case of such a face covering, this snug fit may exacerbate the problem of trapped moisture and having a wet and/or frozen covering touching a user's skin. In addition, if the face covering is used in conjunction with other equipment, such as goggles or sunglasses, the snug fit of the face covering may cause the exhaled air of the user to travel to the area of the other equipment and cause problems for the user, such as fogging of eye coverings (e.g., goggles or sunglasses). Also, a user may remove the covering to dry it and thus it may be more easily lost or misplaced.

Thus, it would be advantageous to have a face covering that improves the venting of exhaled air from the user into the atmosphere while covering an amount of the body that is desired by a user while also resisting unwanted movement or contact with a body part. Such a face covering could advantageously reduce or minimize many problems, such as reducing the accumulation of moisture on the covering, reducing the freezing of moisture on the covering, and reducing contact of any wet or frozen portion of the covering with the user's body, among others. It would also be advantageous to have a face covering that can be easily moved to the side when not in use, connected/disconnected, and connected to other equipment in such a way as to reduce the possibility of loss. If a user were to wear the face covering with eye accessories, such a face covering could advantageously reduce or minimize fogging or freezing or accumulation of moisture of the eye accessories (such as eye protective wear like goggles, sunglasses, etc.). These and other needs are addressed by the various embodiments and configurations of the present invention.

Advantages of the coverings described herein may be obtained while also achieving better protection from the weather and/or sun (e.g. the covering may allow humid air to escape while at the same time providing better coverage of a user's skin than other coverings). Embodiments described herein may be used to protect a user from adverse elements, and may advantageously incorporate a user's wearing of other protective equipment, such as a helmet, goggles, etc. Embodiments disclosed herein may provide a lightweight yet easily maneuverable apparatus, system and method for protecting the face and/or providing comfort for the user. Difficulties in the prior art are overcome in a way that is simple and efficient, while providing better and more advantageous results in various embodiments. Embodiments of the present disclosure provide some illustrative advantages, among others.

The present invention can provide a number of advantages depending on the particular configuration. Advantages described herein as well as other advantages will be apparent from the disclosure of the invention(s) contained herein.

The phrases “at least one”, “one or more”, “or”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C”, “A, B, and/or C”, and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “means” as used herein shall be given its broadest possible interpretation in accordance with 35 U.S.C., Section 112(f) and/or Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The preceding includes a sometimes-simplified summary of the invention to provide an understanding of some aspects of the invention. The summary is neither an extensive nor exhaustive overview of the invention and its various embodiments. It is intended neither to identify key or critical elements of the invention nor to delineate the scope of the invention but to present selected concepts of the invention in a simplified form as an introduction to the more detailed description presented below. As will be appreciated, other embodiments of the invention are possible utilizing, alone or in combination, one or more of the features set forth above or described in detail below. In addition, advantages described herein are illustrative advantages that do not limit the disclosure. Also, while the disclosure is presented in terms of exemplary embodiments, it should be appreciated that individual aspects of the disclosure can be separately claimed.

### BRIEF DESCRIPTION OF THE DRAWINGS

FIG. 1 shows illustrative embodiments of covering devices in combination with a pair of goggles;

FIG. 2 shows illustrative embodiments of covering devices in combination with a pair of goggles;

FIG. 3 shows illustrative embodiments of covering devices in combination with a helmet;

FIG. 4 shows illustrative embodiments of covering devices in combination with goggles and a helmet on a user's head;

FIG. 5 shows illustrative embodiments of covering devices in combination with a helmet;

FIG. 6 shows illustrative embodiments of covering devices in combination with a helmet on a user's head;

FIG. 7 shows illustrative embodiments of covering devices in combination with goggles and a helmet on a user's head;

FIG. 8 shows illustrative embodiments of attachments of covering devices in combination with goggles;

FIG. 9 shows illustrative embodiments of covering devices in combination with goggles;

FIGS. 10A and 10B show illustrative embodiments of attachments of covering devices in combination with goggles;

FIG. 11 shows illustrative embodiments of attachments of covering devices in combination with a goggles strap;

FIG. 12 shows illustrative embodiments of attachments of covering devices;

FIG. 13 shows illustrative embodiments of attachments of covering devices in combination with a goggles strap; and

FIG. 14 shows illustrative embodiments of covering devices.

### DETAILED DESCRIPTION

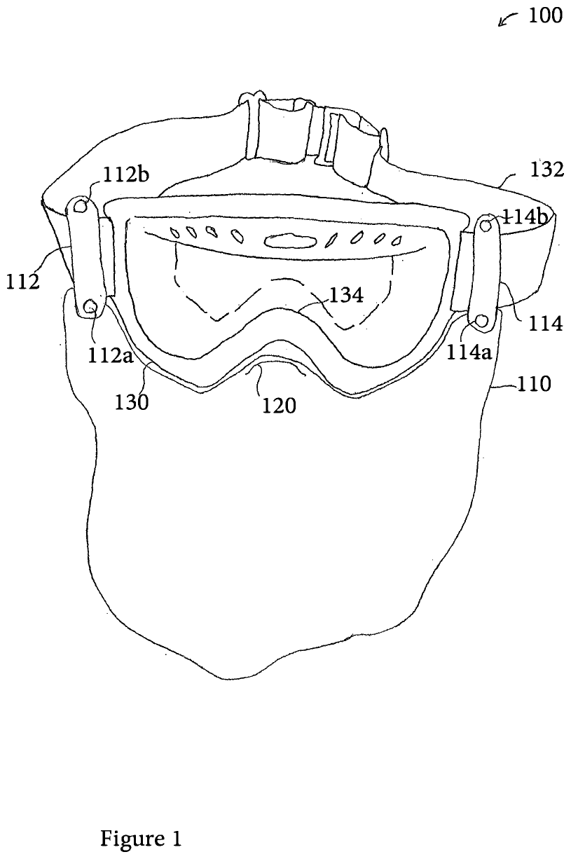

FIG. 1 shows illustrative embodiments of a covering device 100 that includes a face covering 110 with attachment devices 112 and 114. In FIG. 1, an illustrative pair of goggles 130 is also shown. The face covering 110 has a curved portion 120. The attachment devices 112 and 114 may include various points of attachment 112a, 112b, 114a, and 114b that can attach to a strap 132 of the pair of goggles 130.

The covering device as described herein may be referred to as a covering, a covering device, a face covering, an apparatus, a method, and/or a system, among others, and is not limited by such description. It may also be referred to as a protective device, method, and/or system. Embodiments of the description herein also relates to devices for protection and/or coverage of a user's face, neck, and upper torso. The devices, methods, and/or systems described herein can be secured to equipment of a user, such as headgear and/or apparel. The

FIG. 1 shows some embodiments of covering methods, systems, and devices of the present disclosure. In the coverings of the present disclosure, any suitable configuration may be used to attach the covering so that it hangs over a portion of a user's body. For example, any suitable configuration may be used to attach a covering at or around sides of a user's face or head such that the covering is suspended across a user's face and hangs down to cover a portion of a user's body. The covering may be operatively attached to any item at the sides of a user's face or equipment around a user's head (such as a helmet, hat, hood, goggles, glasses, sunglasses, visor, earmuffs, earphones, ear warmers, etc.). For example, the covering may be attached, using any securing means, to an item at the sides of a user's face or equipment around a user's head. There may be multiple attachment points, or different types of attachment methods, systems, or devices, to adjust the configuration of the covering. For example, the different attachment points may enable a user to hang the covering more snugly across the face when attached at certain points and to hang the covering more loosely or even in an uneven configuration when attached at other points on one or both sides of the user's head.

In embodiments, the covering may be convertible between a covering configuration and a hanging configuration. The covering configuration may be where the covering is suspended across a user's face and hangs down to cover a portion of a user's body. The hanging configuration may be where the covering hangs looser than the covering configuration; e.g., the covering may be loosened at one or both sides of the user's head to expose a greater portion of the user's face or neck, or the covering may be detached at one side so that it hangs from the other side of the user's head. In various embodiments, the attachment methods, systems, and devices for attaching the covering to sides of a user's head may be movable and/or removable on the covering itself, and/or at the sides of the user's head. For example, the covering may have movable attachment devices that are clips that can fasten at different points along an edge of the covering. Ends of the clips opposite to the ends attached to the covering can interface to hooks on attachments that are positions at multiple locations at a side of the user's head, for example on a user's helmet. Thus, various different portions of the covering can be attached to various different positions on each side of a user's head. The covering may be large enough to cover a user's entire or partial face and have openings corresponding to the user's eyes (and, optionally, nose and/or mouth), or goggles or glasses, for example. The covering may include vents or slots.

The methods, systems, and devices for converting the covering from a covering configuration to a hanging configuration include any type of methods, systems, or devices that allow for the converting.

Referring again to the embodiments shown in FIG. 1, one or more portions of the covering device 100 (such as an edge or corners of the face covering 110, for example) may be adapted to be affixed to a user's equipment, such as a helmet, goggles, and/or glasses. The covering device 100 including the face covering 110 may be attached to any item on a side of a user's head or face, such as a helmet, glasses (including sunglasses), or a pair of goggles (such as goggles 130 shown in FIG. 1), for example, by attachment devices 112 and 114 at attachment points 112a and 112 b on attachment device 112, and at attachment points 114a and 114b on attachment device 114.

The attachment devices 112 and 114 may be any type of device to hold the face covering 110 to either side of the head or face of a user, or a device on a user's head. The attachment devices 112 and 114 may function to retain the face covering 110 on one side of a user's head or face while releasing the face covering 110 from the other side of the user's head or face. When the face covering 110 is released from one side of the user's head or face, it may hang down from the other side so that the user's head/face is exposed, or also be removed/released from the other side of the user's head or face. For example, the attachments devices 112 and 114 may attach the face covering 110 to something a user is wearing, such as goggles, glasses, sunglasses, ear warmers, a headband, or a helmet, among others. In FIG. 1, the attachment devices 112 and 114 are shown attaching to a strap 132 of a pair of goggles 130.

The covering may be connected to or disposed on or near a front or end edge of a piece of a user's equipment, such as a hat, goggles, a helmet, a hood, sunglasses, etc. Further, the covering may be connected to or disposed on or near a front or end edge of a strap 132 of the goggles 130 (the front or end edge of the strap 132 being portions of the strap 132 that are closer to a face of the goggles that would cover a user's eyes when worn by the user). However, the covering may be connected to or disposed on or near any portion of the goggles 130 and along any part of the strap 132 of the goggles 130. Manners of achieving this may be a permanent or detachable attachment devices, methods, or systems. The permanent or detachable attachments may have a component of a two-part connection assemblage that may have any means of connecting (e.g., press-together, snap-together, hook-together, etc.). The attachment at each side may be the same or different from each other. The attachments may be any type of attachment that would function to attach the face covering 100 to a point (or points) at the user's head (including equipment or accessories on the user's head). Such two-part connection assemblages may be, for example, as shown at points 112a and 114a in FIG. 1.

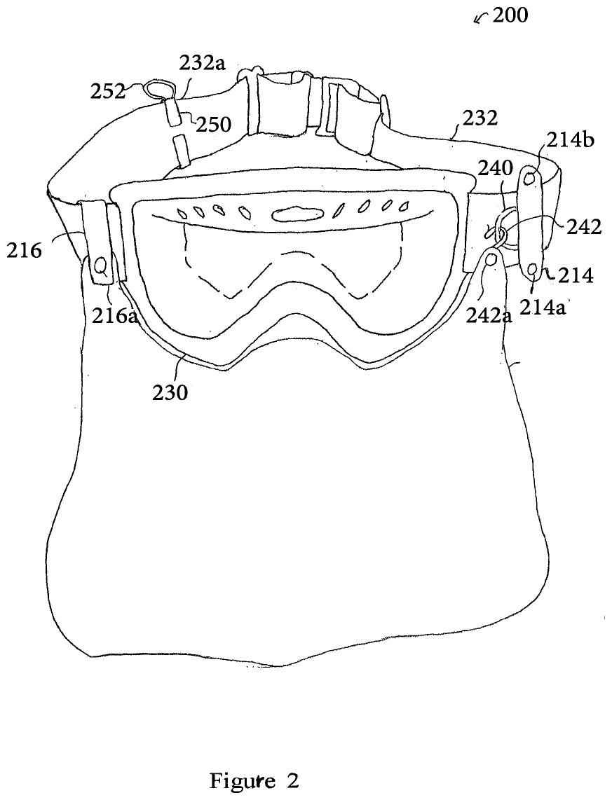

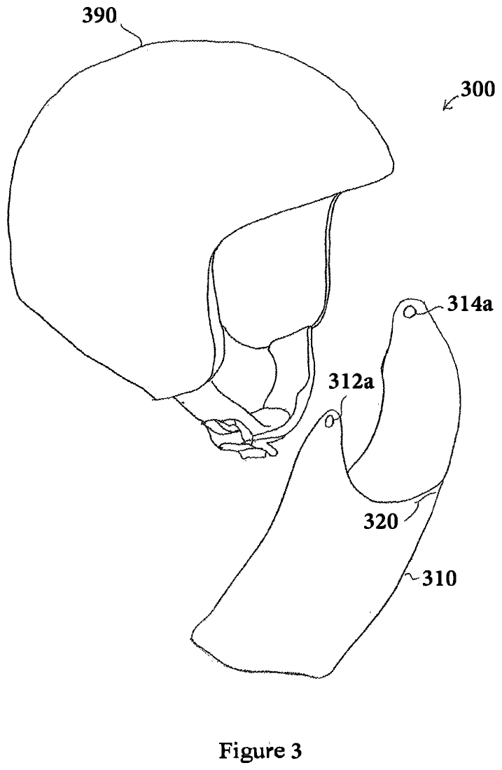

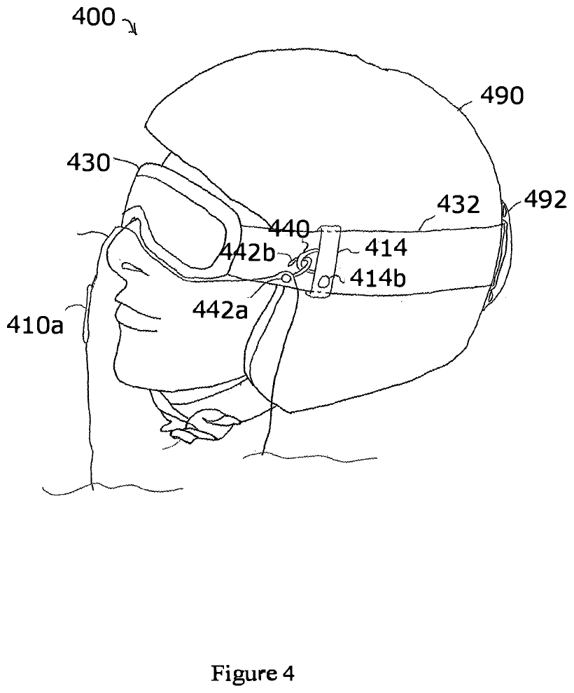

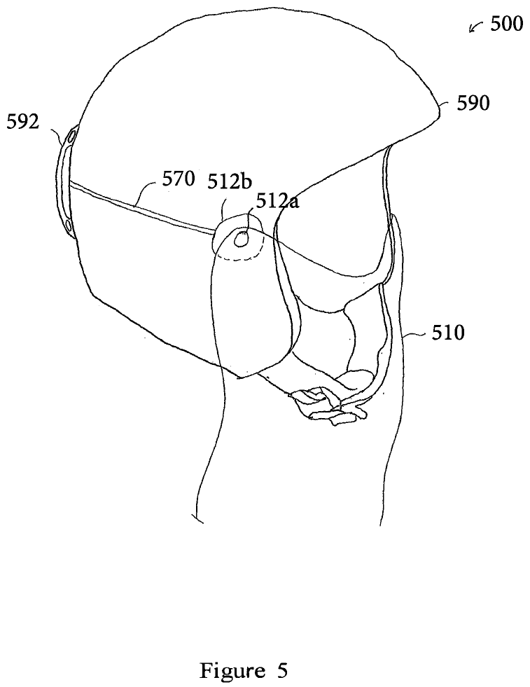

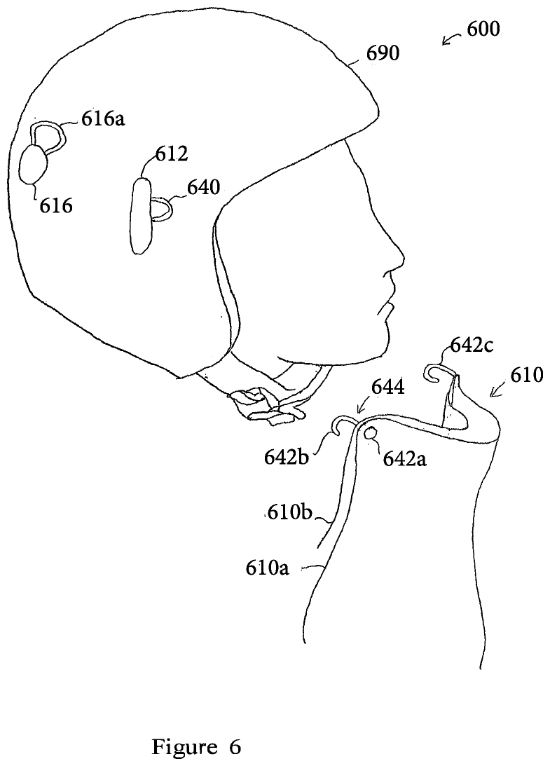

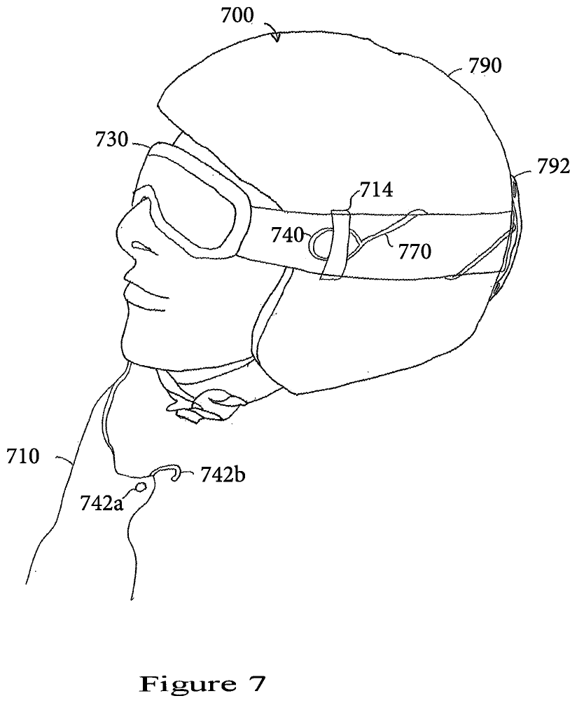

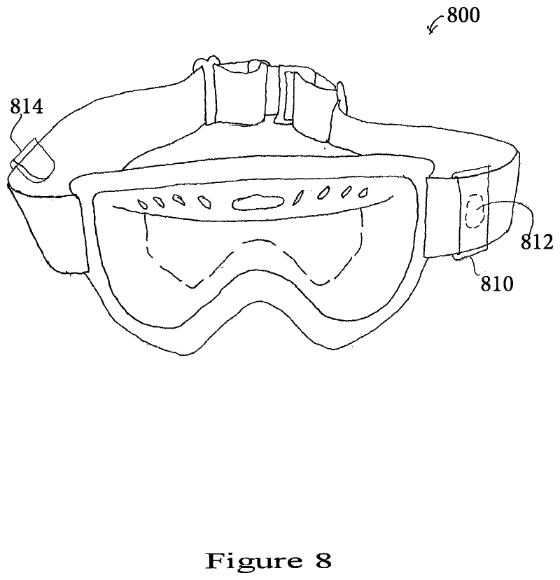

Complementary to each of the two-part connection assemblages may be additional connection pieces 112 and 114, which may be any type of connection device to connect to the goggles 130. For example, the connection pieces 112 and 114 may each be configured such that they are provided on both sides of the goggle strap 132 and such that they have a tacky or textured surface to prevent the connection pieces from moving along the goggle strap 132 after they are placed at a desired position. The additional connection pieces may be have other two-part connection assemblages 112b and 114b to connect them to the goggle strap 132. Further examples of the connection pieces 112 and 114 are shown in FIG. 2 as elements 214 (including 214a and 214b), 216, 240, 242 (including 242a), 216 (including 216a), 250, and 252; FIG. 3 as elements 312a and 314a; FIG. 4 as elements 414 (including 414b), 440, 442a, 442b; FIG. 5 as elements 512a and 512b; FIG. 6 as elements 612, 640, 616, 616a, 642c, 644, 642a, and 642b; FIG. 7 as elements 740, 714, 770, 742a, and 742b; and FIG. 8 as elements 810, 812, and 814; among others.

The components 112 and 114 (including 112a, 114a, 112b, and 114b, in various configurations) may be any suitable connective materials, such as, for example, hook and loop fasteners, closures, snaps, hooks, buttons, tape-type material, reusable sticky-back material, textured material, snap fasteners, pins, VELCRO®, stiff fasteners, soft fasteners, clips, magnets, the type of snap together hook fasteners used in nursing undergarments, etc., and/or a combination of any of these or others). The connections formed by components 112 and 114 (including 112a, 114a, 112b, and 114b, in various configurations) could be fixedly or removably attached to a user's apparel or accessories.

The connections formed by components 112 and 114 (including 112a, 114a, 112b, and 114b, in various configurations) can allow the user to move or remove the covering, and can allow the user to do so quickly and easily. For example, the user may be able to grab the covering (or a portion of the covering) and simply tug at it to release the covering from one or both sides of the user's head (e.g., one side of the goggles strap 132). The size and/or shape of the connection components may advantageously allow for greater ease in the removal, attachment, and/or adjustment of the covering/device while having inhibited capabilities to grasp or move (such as when a user is wearing hand coverings like gloves or mittens, or when the user is concentrating on other actions at a same time as adjusting or removing the covering. The covering may be or may provide a one handed quick connection or release safety connection, while at the same time providing a quick release safety system where the device may be connected or removed by the user instantly with just one hand, or with just one hand that has a glove or covering or other circumstance that inhibits mobility.

The user may easily choose a configuration of the covering and convert it between various configurations. In embodiments, if the covering is damp or wet or covered in snow, the user may more quickly dry the covering by having it in the hanging configuration (e.g., having the covering attached only at one side of a user's face or head, so that it dangles from the one side). The ability to have the covering in a hanging configuration or have the covering in various configurations (such as more loosely attached at the sides to expose more of the face (e.g., portions or all of the nose and/or mouth) may advantageously allow a user to not have to remove the covering.

The covering may also have stitching along each of its edges to prevent fraying or other wear and tear. In addition or alternatively, the covering may have a stiffer portion, such as a wire or wiring or other reinforcement-type material, along one or more of its edges, or portions of its edges and/or within inner areas of the covering to stiffen and stabilize the covering to remain in position over a portion user's face and/or skin.

The covering device 100 can have attachments (e.g., 112a and 114a) on multiple sides/edges and/or corners of the face covering 110, so that the face covering 110 can be rotated to use different attachment points to change the position of the face covering 110, e.g. invert it, flip it around, switch one side for another, switch one edge for another, etc., if part of the face covering 110 becomes wet or damaged. The face covering 110 may also be removable by detaching it at each of the attachment devices 112 and 114, and a new or different face covering 110 may be attached at the attachment devices 112 and 114. The attachments on multiple sides and/or corners may change a look of the face covering 110 or give the face covering various differing shapes/configurations against a user's body. This is shown in FIG. 6, for example.

In addition, the covering may have attachments at a bottom portion of the face covering 110 to attach the bottom to a user's clothing or equipment to keep the covering from moving to a position that is undesirable for the user. The covering may be of a shape and/or size that allows a user to tuck the covering into a jacket or other equipment being worn to keep the covering in a desirable position, such as to keep a bottom portion of the covering inside an opening of a jacket so that the covering covers the neck of a user and does not move around or expose a user's skin when a user is moving or air is blowing.

A liner or multiple liners can be inserted inside the covering (e.g., a fleece liner or a liner formed of another insulated material) to provide insulation and comfort for the sleeper. The liner can be another covering that can add to or replace a covering currently attached. The liner can be added onto a covering currently attached. For example, the liner can be of a different material, shape, and/or configuration (e.g., smaller, larger, having vents, and/or having more layers, among other configurations) for the user to use if the weather conditions change). Additional options to the user may be available by providing various liners that may be exchangeable (e.g., a bright material that may be used for display for safety, a material that incorporates warmer pockets for inserting warming elements, a material that incorporates wires or speakers for connection to electronic devices, a material that contains extra pockets or holes for incorporating electronic devices, etc.).

In further illustrative examples, if the face covering 110 does not lay directly against (e.g., touching) a body part of the user such that there is air between the face covering 110 and the body, then moist air exhaled from a user's nose and/or mouth may enter the air between the face covering 110 and the body and may not be absorbed by the face covering 110, thereby keeping the face covering 110 drier than if the material were in contact with areas of the body. For example, the face covering 110 may have a looser fit next to a user's face than face coverings of the prior art, and such a fit may be achievable due to the designs and systems disclosed herein. This can be advantageous compared to face coverings of the prior art because it can prevent or reduce a wetness of the material. As an illustrative example, various prior art gaiters and balaclavas tightly wrapped around a portion of a user's head and neck so that the material directly contacted a user's nose, cheeks, and mouth, thereby resulting in the material becoming wet with moisture from the user's nose and mouth and becoming uncomfortable for the user. Various embodiments of the present disclosure advantageously address these problems, including the embodiments where the face covering 110 does not lay directly against body part(s) of the user.

In various embodiments, the covering may be of a shape or configuration that can allow for gaps at the sides of the covering (e.g., at sides of a user's neck and/or face) via which moist air can exit to an outside of the covering (the “outside” of the covering can be a side of the face covering 110 opposite to a side that is in partial or full contact with a user's body, which may also be a side that is more in contact with the atmosphere than an opposite side). The material may be selected to more easily be able to form various gaps to air outside, for example by being of a stiffer material or having components incorporated with the material to hold their shape (e.g., stiff wires within the material). Alternatively, the material may be selected to discourage having gaps to the air outside, and the material may be changeable as described herein.

The design or shape of the covering may be such that it improves airflow or ventilation as described herein. The ventilation and/or airflow may be from any side of the covering. The design or shape of the covering may be such that it advantageously improves airflow and breathability as well as reducing and/or preventing fogging to the user's goggles or helmet visor and covers the user's face and skin, which can be beneficial to protect the face and skin while allowing for eyesight in adverse conditions.

Although the face covering 110 in FIG. 1 is shown as a somewhat semicircular shape with various curves, it may be any shape including triangular, curved, square, rectangular, and oblong, among others. It may have some edges longer than others, may have a width that is longer than a height, or a height that is longer than a width. It may have a shape that helps the covering maintain a certain configuration (e.g. be fitted to a shape of a user's face or a user's equipment). It may be made of a material that can help protect or shield a user's face and/or neck (e.g., a firm or hard material). A portion or all of a covering may have any type and extent of structural rigidity to provide any of the embodiments described herein. The covering may have connectors (such as clips, straps, VELCRO®, snaps, hooks, magnets, etc.) at any positions to provide any of the embodiments described herein (e.g., portions or all of the covering may be fastened or unfastened, extended or condensed, folded, rolled, packed, bundled, stuffed, etc.) as desired to change the configuration and/or dimensions of the covering.

Various edges (which may also referred to herein as sides (thus, sides may have various meanings as used herein, such as in any of the x, y, or z-directions) of the face covering 110 may be similar or the same lengths, or different lengths. In various embodiments, sides that extend down along a neck of a user may be longer than the length of a side that extends across the face, e.g., between attachment devices 112 and 114. Portions of the covering may be a size that may be tucked into other clothing, e.g., tucked into a jacket or shirt of a user. The face covering 110 may also have various shapes within the interior of the covering, such as a protrusion or stretched or extra fabric adjacent to where a user's nose and/or mouth and/or chin would be, and/or slits, holes, or other means of ventilation adjacent to where a user's nose and/or mouth may be, among others. The face covering 110 may have various different types of material or stitching within the interior of the covering, such as different type of material or thickness of material adjacent to where a user's nose and/or mouth and/or chin would be. As described herein, the shape and/or size of the covering may provide advantageous effects.

In various embodiments, there may be extra layers of material attached (either removably attached or permanently attached) within the covering. The extra layers may also be called face coverings and liners herein. These extra layers may be of various sizes and shapes, and may or may not be the all the same size and shape. They may be extra layers of material at the nose and/or mouth area (or an area large enough to cover the nose and mouth) that may be moved aside or removed. Extra layers may be all attached (e.g., sewn or otherwise fixedly or removably attached) to one side, edge, and/or area of the covering.

Pockets may be incorporated with the covering. For example, the covering may have a pocket to store items inside of the covering, such as liners, layers, other coverings, a camera, other materials or clothes, food items, toiletries, facial tissues, electronic items, etc. One or more pockets may be on a bottom side of the face covering 110 so that they are tucked into a jacket or other item to keep the items in a safe and warmer position. A pocket may hold a portion of the face covering 110 and/or items associated with the covering. Pocket(s) may also be included in a configuration that is helpful to hold items associated with children, such as global positioning system (GPS) trackers or other tracking or alerting devices. Advantageously, one or more pockets or attachments may be incorporated at a position on the face covering that would be covered by an article of a user's clothing or equipment (e.g., adjacent to a user's chest and under the user's jacket), so that items in the pocket or attached to the face covering would be protected from the weather or outside adverse elements (e.g., snow, rain, etc.), while being readily accessible to the user and safe from falling out. Also, such items may include headphones, speakers, and other electronic devices. In embodiments, a mobile device may be kept in the pocket(s) or attached to the face covering so that a user may interact easily with the mobile device, such as by using voice commands.

Waterproof layer between other layers; for example, six layers of material where the insides can be moved (e.g., flipped, folded, etc.) to be on an interior or exterior of the covering to provide a fresh and/or dry side against the user.

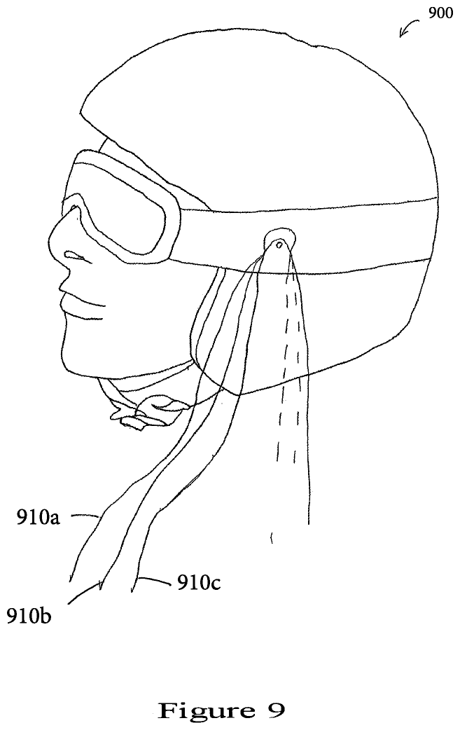

Illustrative examples of these extra layers are shown in FIGS. 6 and 9, for example. The extra layers may function to be moved or removed when they become damp or wet, for example, when a user's breath makes the material damp or wet. As shown in FIGS. 6 and 9, there may be multiple layers of material that are connected at one or more portions of the material, in one more locations, or along one or more edges. As one side gets wet or uncomfortable, the user may flip the material over at the edge to place a fresh side of material against the user's skin, and this may be done multiple times. In embodiments, the various layers may have different designs, and/or be made out of different types of material or have different designs to provide options for the user to choose from.

In various embodiments, the face covering 110 may be reversible, with a different type of material (e.g., a thicker or sheepskin type of material) or a different design on the reversed side. The attachment devices 112 and 114 may be configured to provide the same functionality and/or configuration when the face covering 110 is reversed, or a different type of functionality and/or configuration as described herein.

The material of the covering may be any type of material or combination of material. For example, the face covering 110 may be made out of cotton ribbing, a knit material, leather material, sheepskin material, felt material, neoprene material, cotton material, synthetic material, and natural material, among others, including blends of materials. The covering may be made of any material, such as natural fibers, synthetic fibers, blends, fleece, nylon, cotton, polyester, rip stop material, water-resistant material, and faux materials, among others.

The face covering 110 may be contoured or have an adjustable shape to conform to the nose on the user's face at a nose portion 120 of the face covering 110. A portion of the face covering 110 may be formed to fit over the nose of the user such that an edge of the face covering 110 is adjacent to a lower or bottom portion of the goggles 130. This same edge may also be formed to fit adjacent to a curved lower or bottom portion of the goggles 130, and the form of the edge may take different shapes to be adjacent to other types of objects, such as glasses or sunglasses. For example, as shown in FIG. 1, the face covering 110 may have a shape 120 that corresponds to a nose portion 134 of the goggles. Also, edges of the face covering 110 may be shaped to provide additional comfort or improved wearability to the user, such as having a shape that falls below the eyes while covering the temples and a top of the nose or an area between the eyes.

A configuration of the covering may allow for an edge of the face covering 110 to be placed between a lower portion of the goggles 130 and a user's face to better protect the user's skin from the weather. For example, a user may place the covering in a covering configuration and then slightly raise the goggles 130 off of their face to place the goggles 130 on top of an edge of the face covering 110, or a portion of the edge of the face covering 110.

In the figures described herein, various elements within the figure may not be drawn to scale. Also, the description herein is not limiting and is provided to illustratively describe various exemplary embodiments of the disclosure. The elements described in the figures are not limited by the description.

FIG. 2 shows illustrative embodiments of a covering device 200 in combination with a helmet and a pair of goggles. The covering device 200 includes a face covering 210 with attachment devices 214, 216 and 250. The covering device 200 is illustratively shown attached to goggles 230 having a strap 232.

The attachment device 214 shown in FIG. 2 has points 214a and 214b that may allow the attachment device 214 to be connected to the strap 232. For example, the attachment points 214a and 214b may connect to a structure that extends on the opposite side of the strap 232 from the side of the attachment device 214 that is shown in the figure (such as shown as the side of attachment device 250 in FIG. 2, although the attachment points 214a and 214b are not shown as part of the attachment device 250). Thus, the attachment devices described herein may be slidable along equipment worn by a user, such as the strap 232, or able to detach and re-attach at different positions along the equipment as desired by the user. In other various embodiments, attachment devices may be removably or permanently affixed to equipment of a user. The attachment devices may be configured or designed to attach to the equipment in a manner that they are adjustable by the user. For example, they may be clips that are movable, or have different slots to attach to on the face covering, etc.

The attachment device 214 may have an attachment point 240 to attach a corner or edge of the face covering 210 to the attachment device 214. The attachment point 240 may be a loop having a hook 242b attached to the attachment point 240. Alternatively, the attachment point 240 may be a hook with the loop on the face covering 210. The hook 242B may be permanently or removably attached to the face covering 210 at point 242a.

The materials, designs, and configurations of the attachment devices, including any part of the attachment devices such as the attachment points, are not limited by the description herein.

FIG. 2 shows additional embodiments of an attachment device 250 of the present disclosure. The attachment device 250 may be movable on the strap 232 and may be positioned by the front of the goggles 230 (a “front” may be a portion of the goggles 230 close or on the user's face, or a location on the strap 232 closer to a user's face than a back of the head of the user) to hang the face covering 210 across a user's face, or it may be positioned further back on the strap 232 at position 232a as a point to attach the face covering 210 when not in a covering configuration (e.g., not hanging across a face of a user). For example, the face covering 210 can be attached at both of attachment devices 216 and 250 and in such a configuration, the face covering 210 may be kept away from a user's face. Such a configuration may be useful for when a user wants access to their face for eating, drinking, etc. or if the user does not want the face covering 210 across their face but does not want to entirely remove the face covering 210 or the covering device 200.

Alternatively, attachment devices of the covering device may be positioned anywhere along a user's equipment, including anywhere along a strap of goggles or a helmet. A user may desire to place the attachment devices at positions on a goggles' strap that are closer or correspond to a back side of the user's head. Such a configuration may allow the user to have a face covering that extends along the sides of the user's head so that the face covering covers more area (e.g., covers a user's ears, sides of the neck, etc.

FIG. 3 shows illustrative embodiments of a covering device 300 as assembled together with a helmet 390. In FIG. 3, the covering device 300 includes a face covering 310 with attachment points 312a and 314a. Although the attachment points 312a and 314a are shown as generally circular shaped elements, the attachment points 312a and 314a may be any type of device or configuration (e.g., a hole or slit in the fabric to be attached at a corresponding attachment point on a user's equipment via hanging by a hook, a loop of VELCRO®, a piece of VELCRO®, etc.) and attachment points 312a and 314a may be location at any position on the face covering 310. Additionally, attachment points 312a and 314a may be movable on the face covering 310, or there may be additional attachment points on the face covering 310 so that a user has a choice of where to attach the face covering 310. The face covering 310 may have different types of attachment points that may provide different methods of attachment or different devices to use to attach the face covering 310. The various attachment points that may be on the face covering 310 may be removable, movable, or permanently integrated onto the face covering 310.

A front top edge 320 of the face covering 310 may be positioned against a user's face when the face covering 310 is in a covering configuration. For example, the front top edge 320 may be a side/edge of the face covering that extends between attachment points/devices 312a and 314a. In embodiments, the front top edge 320 may fit snugly over a portion of the user's face, below the user's eyes. The face covering 310 may be positioned in various configurations so that the front top edge 320 can hang lower, such as under a user's eyes and nose, or under a user's eyes, nose, and mouth, or under a user's chin, for example. In a hanging configuration, the face covering 310 may be attached at only one of the attachment points 312a and 314a.

FIG. 4 shows illustrative embodiments of a covering device 400 as assembled together with goggles 430 and a helmet 490 on a user's head. In FIG. 4, the goggles 430 have a strap 432 that is attached to the helmet 490 by feeding through (or being attached through) a strap holder 492 on the helmet 490. The strap holder 492 on the helmet 490 may hold the goggles 430 onto the helmet 490 if the user moves the goggles 430 off of the user's face or if the goggles 430 fall off of the user's face (e.g., if the user is participating in winter sports and the user falls, the strap holder 492 would help prevent the goggles 430 from becoming detached from the helmet 490). If the covering device 400 is attached to the goggles 430 (e.g., on the strap 432), then the covering device 400 would also remain attached to the goggles 430 and therefore to the helmet 490 in the event the goggles are removed from the user's face). This may be advantageous to the user because the user would not have to worry about losing the covering device 400 or misplacing the covering device 400, even if the covering device 400 were not in a covering configuration (e.g., if the covering device 400 were in a hanging configuration).

The covering device 400 can have a face covering attached to a strap 432 of a pair of goggles 430 by attachment device 414 (another attachment device (and/or a permanent fixture of the face covering 410 to the strap 32 or the helmet 490) on the opposite side of the user's head/helmet/goggles strap is not shown in FIG. 4). The attachment device 414 may have a portion 414b that helps the attachment device 414 to stay in a desired position on the strap 432. The portion 414b may have a pin that extends through material of the strap 432, or a protrusion that presses into the strap 432, or be of a stickier material than the rest of the attachment device 414, for example, so that the attachment device 414, once in a desired position for attaching the face covering 410 (e.g., a position that has an edge 320 of the face covering 310 stretched across the user's face in a desired position), is less likely to move along the strap. The attachment device 440 may have a loop (e.g., an eyehole or buttonhole) 440 that attaches the face covering 410 via a hook 442b connected at a point 442a of the face covering 410.

The face covering 410 may have a breathing portion 410a corresponding to a position of a user's mouth and/or nose when the face covering 410 is in a covering configuration. The breathing portion 410a may be any size or shape. The breathing portion 410a may be any type of material or design and may help trap moisture from the user's breathing so that it helps reduce moisture that contacts the remainder of the face covering 410. Also, the breathing portion 410a may have a design that allows for moisture from a user's breathing to escape to the outside air or the other side of the face covering 410, such as vents, holes, flaps, slits, a more loosely woven material, a different type of material, a different thickness of material, etc.

FIG. 5 shows illustrative embodiments of a covering device 500 as assembled together with a helmet 590, which has a strap holder 592. The covering device 500 may have a face covering 100 attached at an attachment device/point 512a/512b and at a secondary attachment device/point (not shown) on the other side of the helmet so that the face covering 510 extends between the attachment device/point 512a and the secondary attachment device/point. The covering device 500 can have a support strap 570 that extends from the attachment device/point 512b to the secondary attachment device/point.

As discussed herein, the attachment device/point 512a/512b and the secondary attachment device/point may be any type of device, material, and configuration, may be any size or shape, and may attach at any locations on the helmet 590 and any locations on the face covering 510. Also, the face covering 510 may be any shape, size, design, material, number of layers, etc. For example, the attachment device/point 512b may be permanently or removably attached to the helmet at a desired position and the attachment device/point 512a may be permanently or removably affixed to the face covering 510. The attachment device/point 512a may interface with the attachment device/point 512b so that the user can easily and quickly attach and detach the face covering 510 at the attachment device/point 512a/512b. In various embodiments, the attachment device/point 512a and 512b may each be a magnetic device that are attracted to one another.

In the figures of this disclosure, elements that are covered by other elements in the figures may be represented by solid lines (such as the portion of the helmet and chin strap of the helmet in FIG. 5 that are covered by the face covering 510 (where the face covering may not be transparent), or elements that are covered by other elements in the figures may be represented by dashed lines (such as the portion of element 512b that is covered by the face covering 510), or elements that are covered by other elements in the figures may be not shown (such as the secondary attachment device/point discussed in relation to FIG. 5). None of these ways of showing elements is limiting and are merely three alternative ways of representing elements that cover each other (e.g., overlap in the figures).

The support strap 570 may function to assist in maintaining positions of the attachment device/point 512a and the secondary attachment device/point. The support strap 570 may also be any type of device, material, and configuration, may be any size or shape, and may attach at any locations on the helmet 590. The support strap 570 can provide tension between the attachment device/point 512a and the secondary attachment device/point to help maintain their positions. The support strap 570 can be permanently or removably or movably attached to any of a user's equipment and/or body, and may be in contact with the equipment for an entirety of the length of the strap (e.g., when it is pulled around equipment such as a helmet) or may be in contact with a user's head or hair for all or a portion of the strap (e.g., if the user is wearing ear muffs or an ear warmer so that the attachment devices/points are at the ear muffs/warmer, and then the support strap 570 could extend around the back of the user's head to be in contact with the user's head and/or hair). For example, the attachment devices/points may be snug loops that fit around arms of glasses worn by the user, with a support strap extending from the one attachment device/point to the other. In such a use, the support strap may advantageously provide tension in a direction opposite from the tension of the face covering stretched between the attachment devices/points to help the attachment devices/points maintain their positions when the face covering is in a covering configuration. Any or all of the support strap may be permanently or removably or adjustably affixed to the user's equipment. The support strap may not extend all the way around a user's head. Alternatively, as discussed herein, the face covering may be used in conjunction with the attachment devices/points without any support strap.

The support strap may take any form, and does not have to be a strap. For example, it can be a wide piece of fabric or a hood, of any material and design. Other examples include the support strap being in the form of a hood that extends to cover most or all of a helmet, with slots or holes where the chin straps of the helmet pass through to hold the hood in place. The attachment points may then be at any location on the hood that is desired, and in any form desired.

FIG. 6 shows illustrative embodiments of a covering device 600 in combination with a helmet 690 on a user's head. In the embodiments of FIG. 6, the covering device 600 includes a face covering having face coverings 610a and 610b and having attachment devices 642a/642b and 642c, together with 612 and 616. As shown in FIG. 6, the attachment device 644 can have various components, such as a connector 642a and a hook 642b. The hook 642b can correspond to a loop 640 that is a component of the attachment device 612, or a loop 616a that is a component of the attachment device 616. In embodiments, if the user desires a more snug fit of the face covering 610, or for the face covering 610 to extend higher on the face, the user may attach the attachment device 644 to the attachment device 616. Also, if the user desires a less snug fit of the face covering 610, or for the face covering 610 to hang lower on the face, the user may attach the attachment device 644 to the attachment device 612. The attachment device 642c may connect to a corresponding attachment device (not shown) on an opposite side of the user's head from attachment devices 612 and 616 that is on the helmet 690.

In FIG. 6, the covering device 600 has a face covering 610 that has multiple layers 610a, 610b. The multiple layers can be any amount of layers and in any shape or configuration. The layers can be attached in any way at the attachment devices, and FIG. 6 shows only an example of some embodiments and is not limiting. The layers may advantageously allow a user to swap or change a layer that is against the body to obtain a dry layer.

For example, in FIG. 6, a layer 610a is further away from the user's body than a layer 610b (a first configuration), and the layer 610a may be referred to herein as a front layer and the layer 610b may be referred to as a back layer in the configuration shown in FIG. 6. Using the original configuration of layers 610a and 610b as an example, if a user is wearing the covering device 600 as shown in FIG. 6, a side of the layer 610b that is against the user's body (e.g., nose and/or mouth may become damp or wet from the user's breath. The user may flip the layer 610a in an almost 360 degree motion (to achieve a second configuration) so that the front side of layer 610a is against the back side of layer 610b, the back side of layer 610a is against the user's body (e.g., nose and/or mouth), and the front side of layer 610b is further away from the user's body than the layer 610a so that the front side of layer 610b is exposed to the outside/air. In the second configuration, the layer 610b is further away from the user's body than the layer 610a. Thus, although a side of the layer 610a may become wet from moisture contacting the covering device 600 (such as a user doing snow sports) in a first configuration, the user may advantageously obtain a drier layer against their body by changing the layers into a second configuration.

The devices illustrated in FIG. 6 are not limiting, and any devices may be used. Although one type of attachment is shown in FIG. 6, other types of attachments may be used, such as one where the layers are removable/detachable from the attachment device to be rearranged by the user. As shown in FIG. 6, if the user moves the layers from the first configuration to the second configuration, the layers may be in a twisted shape at the attachment points (e.g., have a twisted shape at connector 642a). However, other types of attachment devices may be used that would reduce or eliminate any twist in the material of the face coverings, such as one that may let the face coverings rotate freely, such as one having a string or rope that attaches to the corners of the face coverings so that the twist is in the string or rope. Alternatively, the string or rope may be connected by a device that allows the string or rope to rotate freely to avoid any twist. Although two layers are shown in FIG. 6, this also is not limiting. There may be one layer that separates into different layers, or there may be three, four, five, or more layers.

In embodiments, the multiple layers may be advantageous because if the covering device includes multiple layers, and one or more of the layers contains a waterproof material, as the user is wearing the covering device the layer closest to the user's mouth and/or nose (e.g. 610b in the first configuration) may become damp or wet from moisture from the user's mouth and/or nose, and a layer that is furthest from the user's body and exposed to the outside weather elements (e.g., 610a in the first configuration) may also become damp or wet due to snow or rain or other moisture in the outside air. However, the layer that is furthest from the user's body may become damp/wet only on a side that is exposed to the outside air. Thus, when a user wants a drier layer against the body, the user may flip the outside layer to become the inside layer (e.g., to achieve a second configuration such as by flipping the layer 610a so that the front side of layer 610a is against the back side of layer 610b, the back side of layer 610a is against the user's body (e.g., nose and/or mouth), and the front side of layer 610b is further away from the user's body than the layer 610a). Therefore, although one side of the layer that was exposed to the outside weather conditions in the first configuration became damp/wet from moisture in the air, this wet side of the layer is now facing the back side of a different layer, and the dry side of the layer that was exposed to the outside weather conditions in the first configuration is now against the user's body (e.g., nose and/or mouth), advantageously providing the user with fresher and/or drier material against the body.

FIG. 7 shows illustrative embodiments of a covering device 700 in combination with goggles and a helmet on a user's head. In embodiments, the covering device 700 can include a face covering 710, a first connecting piece 742a and 742b that connects to a second connecting piece 740 that is attached to a goggles attachment 714, and the goggles attachment 714 may also be connected to a supporting strap 770. The supporting strap 770 may, for example, extend around the strap of the goggles 730. The supporting strap may be made of any material or combination of materials, and may be stiff or stretchy or have a tacky texture, for example, so that it stays in a desired position on the goggles strap. The attachments may be held in place on the goggles strap (or other piece of user's equipment, such as a helmet) by any other means or devices. For example, the attachment(s) can have a pin that threads through the goggles strap to maintain a position of the attachment(s). A different design or method may be used on different sides or positions of the goggles strap or other user's equipment. The goggles 730 may be worn by the user on a helmet 790. The helmet 790 may have a goggles strap holder 792, though which the strap of the goggles 730 is positioned through the goggles strap holder 792 to keep the strap in place and keep the goggles 730 connected to the helmet 790 even when the user moves the goggles 730 to different positions. Although only one side of the attachment of the face covering with the attachment devices is shown attaching to the goggles strap in FIG. 7, similar elements may be present on the other side of the user's head.

The user may wear the covering device 700 such that the face covering 710 stretches tight across the user's face directly underneath the goggles 730 (e.g., slightly overlapping an edge of the goggles 730 such that the face covering 710 is in between a portion of the goggles 730 and the user's face, or adjacent to the goggles 730 against the user's face). If the face covering 710 is stretched tight, it may put tension on the goggles attachment 714, causing the goggles attachment 714 to move on the strap of the goggles 730 towards the user's face. However, advantageously, the supporting strap 770 can prevent the goggles attachment 714 from moving on the strap of the goggles 730 by being stretched in an opposite direction on the strap of the goggles 730 from the face covering 710. Although illustrative embodiments of the covering device 700, including the supporting strap 770 and the goggles attachment 714, are shown in FIG. 7, these embodiments are not limiting and the covering device 700 can take any shape or form. The materials of the covering device 700 may be any type of material or materials and any design or configuration.

FIG. 8 shows illustrative embodiments of attachments of a covering device 800 in combination with goggles. In FIG. 8, attachment devices 810 and 814 are shown attached to a pair of goggles. As shown in FIG. 8, the attachment devices 810 and 814 may be made in such a configuration that they are removably attached to a strap of the goggles. The attachment devices 810 and 814 may be flexible or hard, and may have any type of texture. For example, attachment devices 810 and 814 may be made of a rubber, silicone, plastic, metal, or a combination of materials. The attachment devices 810 and 814 may be configured so that they slide along the strap of the goggles, or so that they stay in a certain position on the strap of the goggles. As shown in FIG. 8, the attachment devices 810 and 814 may not be connected on a backside of the strap of the goggles. This may allow for the attachment devices 810 and 814 to be removed and reattached to the strap. The attachment devices 810 and 814 may each have an attachment point (e.g., at area 812 shown only on attachment device 810). The attachment points may attach a face covering in any configuration.

FIG. 9 shows illustrative embodiments of a covering device in combination with goggles. In FIG. 9, the covering device 900 has multiple layers of face coverings 910a, 910b, and 910c. The multiple layers may each be attached, separately or together at attachment points to the sides of the user's head, e.g., attached to equipment on the sides of the user's head. In various embodiments and configurations, the layers 910a, 910b, and 910c can be changed to different locations in the covering device so that at times 910a may be closest to a user's face, and at other times 910b may be closest to a user's face, and still other times 910c may be closest to a user's face. Also, layers may be removed and added as needed. For example, layers 910a and 910b may be removed, leaving only layer 910c as the covering device.

The layers may be of different materials and/or shapes, or all the same materials and shapes. The layers may have different designs. For example, one or more of the layers may have multiple layers that are affixed to one another to create a multi-layer layer. For example, a windproof (or wind resistant) and/or waterproof or water-resistant layer or water absorptive layer sandwiched between two soft layers, so that a user's breath is absorbed into the middle layer but the user still has soft layers to touch/feel against the user's body. There may be multiple multi-layer layers that may be placed in various configurations, for example as described in FIG. 6 to provide the user with fresher and/or drier layers against the user's body. For example, each of layers 910a, 910b, and 910c may be multi-layer. One or more of the layers may have a special design, such as a hunter's orange color, that the user can expose as desired. The layers may have different ornamental designs so that the user can change the look of the face covering. If the layers can be removed and reattached at the attachment points, the user may easily change the type and/or look of the covering device, or change one or more of the layers to be a cleaner and/or newer and/or drier layer.

The layers may have different functionality. The layers may include pockets to hold items. The layers may include holes or slots to attach attachment devices or other devices or items. One or more of the layers may have different properties that a user may desire for differing weather conditions. For example, one or more layer may be thicker and/or made of a multi-layer configuration, while one or more other layers are made of a lighter and/or thinner material. The layers may have a shape or configuration that makes it easier for them to stay in a desired location, such as a clip or clips along a corner or edge of the layer to fasten the layer to an article of clothing worn by the user. The devices, systems, or methods of connecting the covering in manners that help the covering maintain a desired position may use any method to do so. For example, the face covering may have various buttonholes so that an attachment device may be moved to different positions on the face covering by inserting a part of the attachment device through the buttonhole. Embodiments of the covering device may include safety features to improve the safety of the covering device when using the device, such as break-away attachments, etc.

Illustrative materials that may be included in the covering devices are not limited and include fleece, thermal fleece, nomex fleece, polar fleece, cotton, polyester, spandex, rubber, plastic, synthetic material, natural material, organic material, neoprene, nylon, wool, silicone, acrylic, silk, polypropylene, stretchy material, stiff material, and material blends.

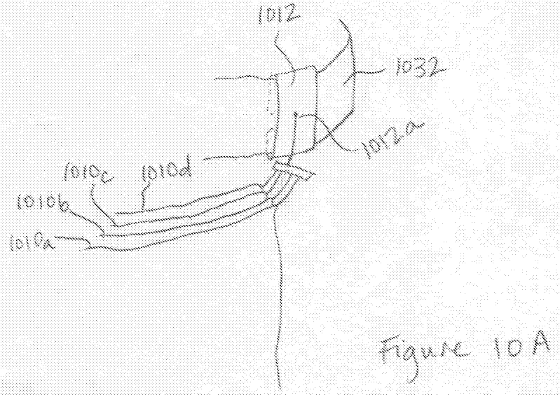

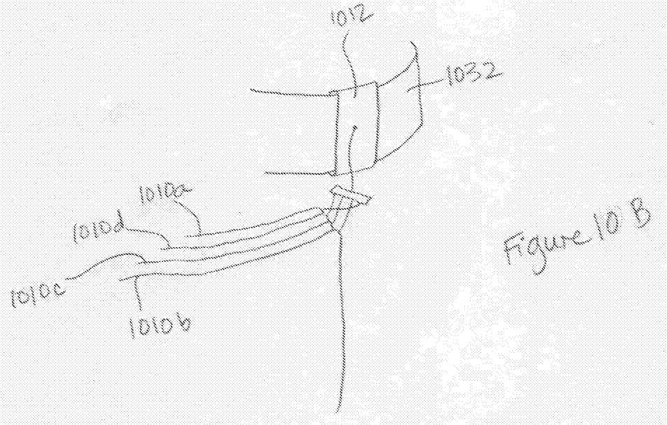

FIGS. 10A and 10B show illustrative embodiments of attachments of covering devices in combination with goggles. In the embodiments of FIGS. 10A and 10B, the covering device includes a face covering having face coverings 1010a, 1010b, 1010c, and 1010d and having attachment devices 1012 including 1012a attached to goggles strap 1032. In embodiments, if the user desires a snugger fit of the face covering, or for the face covering to be suspended higher on the face (e.g., by having more tension between attachment devices/points, the user may attach the attachment device 1012 on the goggles strap 1032 at a position further towards the back of the user's head. As shown in FIG. 10A, the attachment device 1012 may have a configuration that allows easy removal from a goggles strap 1032 and easy adjustment along the goggles strap 1032 so that the attachment device 1012 can move closer to a user's face or further from the user's face (e.g., towards the back of a user's head). In some embodiments, the attachment point 1012a may be adjustable so that it can be shortened or lengthened as desired by a user. These embodiments, among others, may advantageously allow a user to easily adjust covering devices as described herein to have a desired fit. Although only one attachment device 1012 is shown in FIGS. 10A and 10B, a corresponding one (that may be the same or different) may be disposed on another side of the covering device).

In FIGS. 10A and 10B, the covering device has face coverings having multiple layers 1010a, 1010b, 1010c, and 1010d. The multiple layers 1010a, 1010b, 1010c, and 1010d can be any amount of layers and in any shape or configuration. The layers can be attached in any way at the attachment device 1012a and 1012 and in any way to a side of a user's head (e.g., to a goggles strap as shown in FIGS. 10A and 10B as goggles strap 1032). FIGS. 10A and 10B, like the other figures described herein, show only examples of some embodiments and are not limiting.

The layers 1010a, 1010b, 1010c, and 1010d may advantageously allow a user to swap or change a layer that is against the body to obtain a dry layer. For example, as shown in FIG. 10A, layers 1010a, 1010b, 1010c, and 1010d are arranged in that order respectively as furthest from a user's face to closest to a user's face. Thus, a side of layer 1010a that is furthest from the user is a side that is most exposed to outside air, and as such it may be exposed to snow, wet, and cold conditions, thereby becoming wet and/or cold. However, a side of layer 1010a that is closest to the user's face can be protected from the outside air because it is against layer 1010b, and thus it is protected from becoming wet/damp and/or cold (e.g., especially if the layer 1010a includes a waterproofing or water-resistant material). In addition, regardless of the outside air conditions (e.g., wetness or cold), a side of layer 1010d that is closest to the user's face (including, for example, nose or mouth, although it may merely be against a user's skin) can become uncomfortably wet or damp due to moisture from a user's breath and/or nose and/or sweat from the user's skin. Therefore, advantageously, a user may flip layer 1010a over to achieve a configuration as shown, for example, in FIG. 10B.

In FIG. 10B, layer 1010a is flipped over so that layers 1010b, 1010c, 1010d, and 1010a are arranged in that order respectively as furthest from a user's face to closest to a user's face. Thus, the side of layer 1010d that was closest to the user's face is now (due to layer 1010a being flipped or rotated over the other layers) between layers 1010a and 1010c so that it is not directly adjacent to the user's face/skin. With layer 1010a being flipped, the user now has the side of layer 1010a that was against layer 1010b (in FIG. 10A, prior to the flipping of layer 1010a) against the user's skin and/or face. Thus, the side of layer 1010a that was formerly (in FIG. 10A) furthest from the user and exposed to outside air is now against (e.g., directly adjacent and/or mostly in contact with) layer 1010d. In the configuration of FIG. 10B, the user has the side of layer 1010a that was (prior to the flip) protected by being against layer 1010b now against the user's skin (which may include the face), with a side that was more exposed to any weather elements than other parts of the face covering layers now against layer 1010d so that the user does not feel any dampness of the side of layer 1010a formerly exposed to the weather elements after the flip. This may be even more advantageous if the layer 1010a (and/or other layers) has a water-resistant or water-proof element that allows one side (or part) of the layer to stay dry even if another side is exposed to moisture.

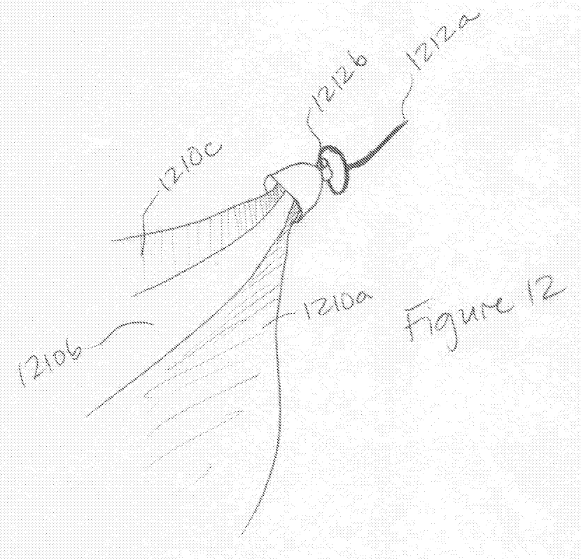

As shown in FIGS. 10A and 10B, if the attachment device 1012 (including any part of the device that extends from the attachment point (e.g., at a goggles strap 1032) to the face covering (e.g., layers 1010a, 1010b, 1010c, and 1010d)) has a configuration that enables easy rotation of the layers, then a user may easily flip any layers to achieve a desired configuration. Thus, the attachment devices and attachment points described herein include any type of devices, methods, and systems that allow a user to flip the layers and place the covering device in a configuration that is desired. For example, the attachment point may be some device that easily swivels, such as a tassel type of attachment as shown, for example, in FIG. 12.

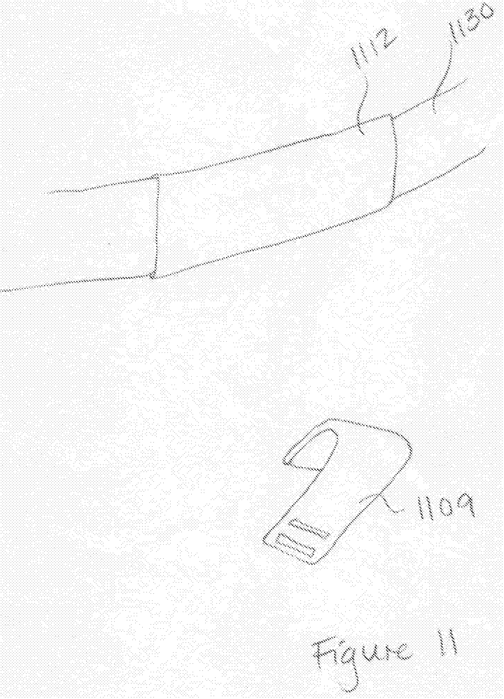

FIG. 11 shows illustrative embodiments of attachments of covering devices in combination with a goggles strap. In the embodiments shown in FIG. 11, the attachment device 1112 can extend along a goggles strap 1130 such that it has an edge that is wider than an attachment hook 1109. In the embodiments shown in FIG. 11, the attachment hook 1109 can be easily slipped over the attachment device 1112 at various points so that a user can adjust where (e.g., how far from the user's face) the attachment hook 1109 is attached to the attachment device 1112 along the goggles strap 1130. In embodiments, the attachment device 1112 has a gap or space between a top potion of the attachment device 1112 and the helmet (the top portion being a portion of the attachment device 1112 closer to a top of a user's head) that assists in the hook 1109 being able to slide between the attachment device 1112 and whatever a user is wearing on the inside of the goggle strap 1130, such as a helmet (not shown in FIG. 11). The attachment device 1112 and the hook 1109 can have any configuration that allows for the hook to attach to the attachment device 1112, and can be made of any material. For example, the attachment device 1112 and/or the hook 1109 may be made of a material that has an amount of friction between the attachment device 1112 and the hook 1109, so that the hook stays in place on the attachment device 1112. Also, the attachment device 1112 may be made of a material or have dimensions that allow for the attachment device 1112 to maintain a desired position on the user's goggle strap 1130 (e.g., extend far enough along the strap that the pressure of the strap against the side of a user's head (e.g., against a helmet) reduces a likelihood that the attachment device 1112 will undesirably shift position along the strap 1130; and/or have a rough area or a gripping area/configuration where the attachment device 1112 is in contact with the goggles strap 1130 along a backside of the goggles strap 1130 (e.g., on a side of the goggles strap 1130 closest to the user's head; and/or be made of a material that increases friction between the attachment device 1112 and the goggles strap 1130; etc.). Material of a face covering (not shown) or various layers of a face covering (not shown) may be attached to the attachment device 1109.

FIG. 12 shows illustrative embodiments of attachments of covering devices. For example, FIG. 12 shows an attachment device 1212b that has a configuration similar to a tassel-type device with layers 1210a, 1210b, and 1210c, attached within the tassel-type connector of the attachment device 1212b, and the tassel-type connector being rotatably connected to a lanyard or extension 1212a, so that the attachment device 1212b can easily rotate to flip the layers 1210a, 1210b, and 1210c, as desired by a user.

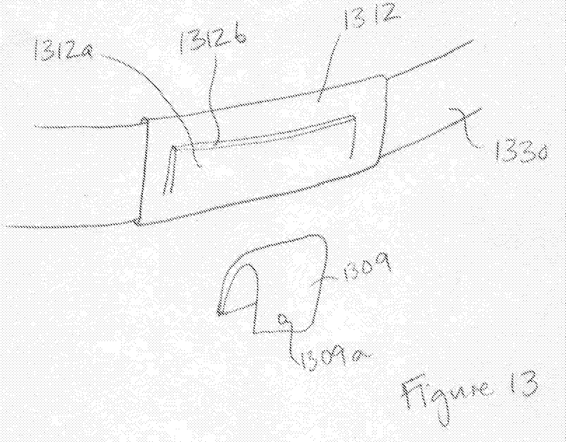

FIG. 13 shows illustrative embodiments of attachments of covering devices in combination with a goggles strap. In the embodiments shown in FIG. 13, the attachment device 1312 can extend along a goggles strap 1330 such that it has an edge that is wider than an attachment hook 1309. The attachment hook 1309 can have any configuration to attach a face covering, or material of a face covering, such as a hole 1309a as shown in FIG. 13. For example, the tassel-type connector shown in FIG. 12 (e.g., the string or lanyard 1212a) may be attached to the hole 1309a shown in FIG. 13.

In the embodiments shown in FIG. 13, the attachment hook 1309 can be easily slipped over a ledge 1312a having a gap 1312b in the attachment device 1312 at various points along the gap 1312b so that a user can adjust where (e.g., how far from the user's face) the attachment hook 1309 is attached to the attachment device 1312 along the goggles strap 1330. In embodiments, the attachment device 1312 can have only a ledge or a gap as part of the attachment device 1312 that assists in the hook 1309 being able to slide into the ledge and/or gap of the attachment device 1312. The configuration of the attachment device and any ledge, gap, or other design is not limited by the description herein, and includes any configuration that allows a user to attach a face covering to the attachment device. The attachment device 1312 and the hook 1309 can have any configuration that allows for the hook to attach to the attachment device 1312, and can be made of any material. For example, the attachment device 1312 and/or the hook 1309 may be made of a material that has an amount of friction between the attachment device 1312 and the hook 1309, so that the hook stays in place on the attachment device 1312.

Also, the attachment device 1312 may be made of a material or have dimensions that allow for the attachment device 1312 to maintain a desired position across a user's face and/or on the user's goggle strap 1330 (e.g., extend far enough along the strap that the pressure of the strap against the side of a user's head (e.g., against a helmet) reduces a likelihood that the attachment device 1312 will undesirably shift position along the strap 1330; and/or have a rough area or a gripping area/configuration where the attachment device 1312 is in contact with the goggles strap 1330 along a backside of the goggles strap 1330 (e.g., on a side of the goggles strap 1330 closest to the user's head; and/or be made of a material that increases friction between the attachment device 1312 and the goggles strap 1330; etc.). Material of a face covering (not shown) or various layers of a face covering (not shown) may be attached to the attachment device 1309. For example, the attachment device can include a strap connecting it to another attachment device (not shown) on another side of the user's head, as in element 770 of FIG. 7. Any face covering described herein may also be configured to extend entirely around a user's body so that only one attachment device is used to attach the covering at a side of a user's head.

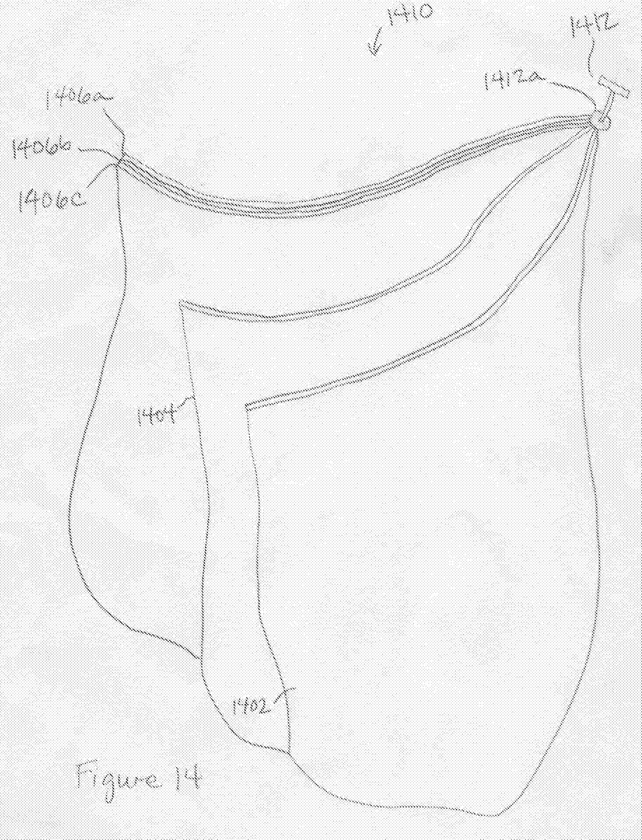

FIG. 14 shows illustrative embodiments of covering devices 1410. In FIG. 14, the covering device has multiple layers 1402, 1404, and 1406a/1406b/1406c that are attached at attachment point 1412a to attachment device 1412. The layers 1402, 1404, and 1406a/1406b/1406c can rotate if desired by the user to have any of layers 1402, 1404, and 1406a/1406b/1406c be closest to a user's body. One or more of the layers 1402, 1404, and 1406a/1406b/1406c can have a multi-layer configuration, and an exemplary multi-layer configuration is shown as layer 1406a/1406b/1406c. In layer 1406a/1406b/1406c, each of the layers 1406a, 1406b, and 1406c, can have any shape and be made of any material (including combinations of materials). In embodiments, layers 1406a and 1406c can be made of a soft material that would be desirable by a user to have against the skin of the body (e.g., a fleece material), while layer 1406b can be made of a waterproof or water-resistant material so that even if one of layers 1406a and 1406c becomes damp, the other of layers 1406a and 1406c will stay dry. Thus, a multi-layer configuration such as 1406a/1406b/1406c can be advantageous by providing a user a dry layer to have against the skin when layers are rotated, even if one of the layers 1406a and 1406c becomes wet.

What is claimed is:

1. A device, comprising:
a first attachment removably connected at a side of the head of a user to a piece of equipment worn by the user;
a second attachment connected at another side of the head of the user to the piece of equipment; and
a flexible covering extending between the first attachment and the second attachment.

2. The device according to claim 1, wherein the second attachment removably connects to the piece of equipment.

3. The device according to claim 1, wherein the piece of equipment is goggles.

4. The device according to claim 1, wherein the piece of equipment is a helmet.

5. The device according to claim 1, wherein the piece of equipment is goggles, wherein the user is also wearing a helmet, and wherein the goggles are worn by the user with the helmet.

6. The device according to claim 1, wherein at least one of the first attachment and the second attachment removably connect to the covering.

7. The device according to claim 1, wherein the piece of equipment is a hat.

8. The device according to claim 1, wherein the piece of equipment is a pair of glasses.

9. The device according to claim 1, wherein, when the first attachment comprises a first portion and a second portion, and
wherein when the first attachment is disconnected from the side of the head of the user then the first portion of the first attachment remains connected to the piece of equipment and the second portion is not connected to the piece of equipment.

10. The device according to claim 9, wherein when the first attachment is disconnected then the second portion remains connected to the flexible covering.

11. The device according to claim 1, wherein each of the first attachment and the second attachment removably connect to the covering.

12. The device according to claim 1, wherein the flexible covering is multiple layers having each of the layers in the multiple layers connected to the first attachment and the second attachment.

13. The device according to claim 1, wherein the flexible covering is a fabric.

14. The device according to claim 1,
wherein the plurality of layers is a first layer and a second layer,
wherein in a first configuration, a second side of the first layer is directly adjacent to a first side of the second layer, and
wherein in a second configuration, a second side of the second layer is directly adjacent to a first side of the first layer.

15. The device according to claim 1,
wherein the plurality of layers is a first layer, a second layer, and a third layer,
wherein in a first configuration, a second side of the first layer is directly adjacent to a first side of the second layer and a second side of the second layer is directly adjacent to a first side of the third layer, and
wherein in a second configuration, a first side of the first layer is directly adjacent to a second side of the third layer.

16. The device according to claim 1,
wherein a first layer of the multiple layers has a first side and a second side,
wherein a second layer of the multiple layers has a first side and a second side,
wherein in a first configuration, the second side of the first layer is directly adjacent to the first side of the second layer and at least one layer is closer to the second side of the second layer than the second side of the first layer, and
wherein in a second configuration, the at least one layer is between the second side of the second layer and the first side of the first layer.

17. A method, comprising:
connecting a first attachment at a side of the head of a user to a piece of equipment worn by the user;
connecting a second attachment at another side of the head of the user to the piece of equipment;
wherein at least one of the first attachment and the second attachment removably connects to a covering, and
wherein the covering extends between the first attachment and the second attachment.

18. The method according to claim 16, wherein the second attachment removably connects to the piece of equipment.

19. The method according to claim 16, wherein at least one of the first attachment and the second attachment removably connect to the covering.

20. The method according to claim 16,
wherein, when the first attachment comprises a first portion and a second portion, and
wherein when the first attachment is disconnected from the side of the head of the user then the first portion of the first attachment remains connected to the piece of equipment and the second portion is not connected to the piece of equipment.
